(19) United States
(12) Patent Application Publication
(10) Pub. No.: US 2010/0062993 A1
(43) Pub. Date: Mar. 11, 2010

(54) Adenosine Nucleotides as Dietary Supplements and as Agents in the Prevention of Cancer and the Metastasis Thereof

(75) Inventor: Yochanan R. Bulka, Lakewood, NJ (US)

Correspondence Address: LIFE SCIENCE LABORATORIES, INC., 170 N. OBERLIN AVE, UNIT 26, LAKEWOOD, NJ, 08701, US

(73) Assignee: Yochanan R. Bulka, Lakewood, NJ (US)

(21) Appl. No.: 12/208,873

(22) Filed: Sep. 11, 2008

(51) Int. Cl. A61K 31/7076; A61P 35/04

(52) U.S. Cl. 514/46

(57) ABSTRACT

It is the embodiment of this invention that Methylthioadenosine and its pharmaceutically acceptable salts as dietary supplements are more efficient, more economical and better tolerated than conventional existing dietary supplements such as S-Adenosylmethionine in the promotion of normal cell growth. It is a further embodiment of this invention that Methylthioadenosine and its pharmaceutically acceptable salts as dietary supplements are more efficient, more economical and better tolerated than conventional existing dietary supplements such as S-Adenosylmethionine in the destruction of aberrant cells that can cause various cancer. It is a further embodiment of this invention that Methylthioadenosine and its pharmaceutically acceptable salts as dietary supplements are more efficient, more economical and better tolerated than conventional existing dietary supplements such as S-Adenosylmethionine in the prevention of cancer cell metastasis

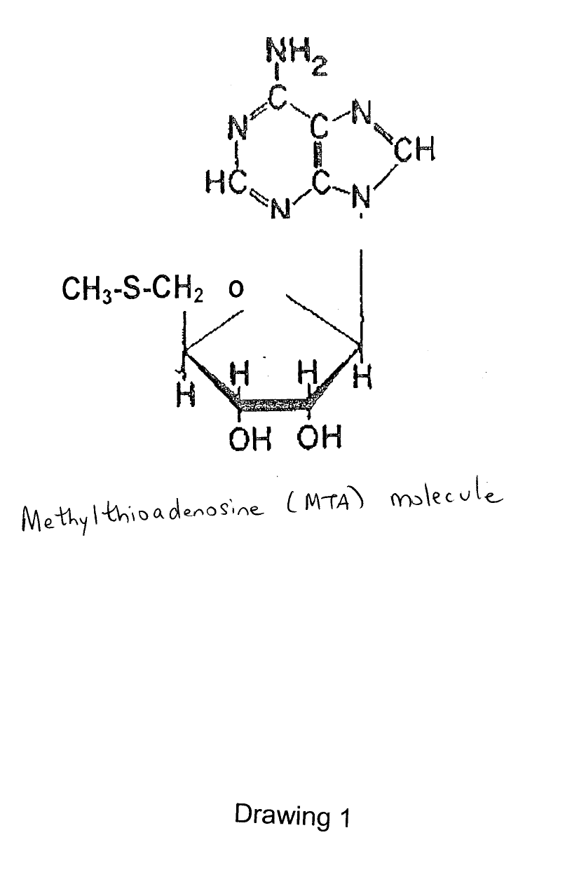

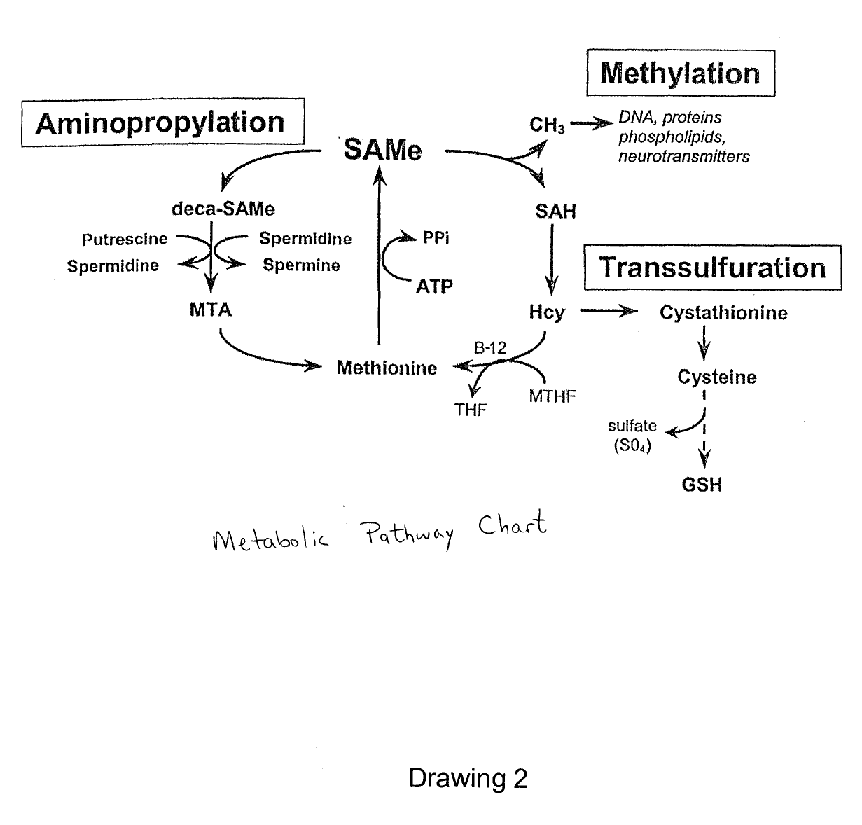

### BRIEF SUMMARY OF THE INVENTION

(MTA) also known as VitaminL2, methylthioadenosine or 5-deoxy-5-methylthioadenosine is a natural metabolite of the amino acid methionine via (SAMe) S-adenosylmethionine metabolism.

SAMe is a highly successful dietary supplement. It has many applications with the most popular being, mood enhancement via the transmethylation pathway and enhanced mobility via the transulfuration metabolic cycle.

SAMe also promotes cellular integrity and normal cell growth via the aminopropylation pathway! MTA is an integral component of this process. Conventional science to date attributes part of SAMe effectiveness against cancer and its metastasis, to be a result of the possible inhibition of cellular demethylation via the transmethylation process. MTA cannot be a contributing factor in this regard as it is not a methyl donor! On the other hand, aminopropylation, to which MTA is a component, is believed to account for as much as one-third of SAMe metabolism.

It is therefore astonishing that MTA, in experimental results on mice implanted with tumor cell linings, was 10 times more efficacious than SAMe. Part of the pharmacodynamics is surely that MTA is toxic to many cancer cells that are deficient in the enzymes that are needed to process it. Yet this 10:1 efficacy ratio is inexplicable under the above suppositions!

MTA is more tolerable as it does not need the 50% acid stabilizing salts. It is more therapeutically cost effective as well. This point is a standalone discovery as well!!

Therefore, MTA is a cost effective-more tolerable dietary supplement, which promotes normal cell growth, and is an agent in the prevention of cancer and its metastasis.

### DRAWINGS

### Listing of Drawings

[Drawing 1] Methylthioadenosine (MTA) Molecule

[Drawing 2] Metabolic Pathway Chart

### DESCRIPTION

5-deoxy-5-methylthioadenosine also known as Methylthioadenosine (MTA) or Vitamin L2 has a chemical structure as shown in Drawing 1. Many of its properties have been elucidated by Schlenk, Stramentinoli etc. in scientific literature.

MTA is a natural metabolite of the methionine cycle via the ubiquitous S-adenosylmethionine (SAMe). Drawing 2 describes the three metabolic pathways of SAMe: Transmethylation, Transulfuration and Aminopropylation. SAMe is now a highly successful nutritional supplement in the USA under the aegis of the Dietary Supplement and Health Education Act of 1994 (DSHEA). The first two pathways lead to mood enhancement and increased mobility respectively.

It should be noted that SAMe has shown efficacy in the treatment of cancer as it inhibits cellular demethylation, one key factor in this disease. This phenomenon is attributed to transmethylation, a direct mechanism of action that cannot be attributed to MTA metabolism as MTA is not a Methyl donor.

Aminopropylation plays a central role in cellular function in which MTA is a key element. Its metabolism and pharmacokinetics and pharmacodynamics are still being extensively studied. The conventional paradigm as expressed in several research articles such as those by Guildori and Sramentinoli was that this third route may account for as much as one third of SAMe metabolism.

Various very important uses for MTA have been discovered and patented.
1. U.S. Pat. No. 4,454,122 Giorgio Stramentinoli, et al. Adenosine Derivatives of Anti-inflammatory and Analgesic Activity, and Therapeutic Compositions which contain them as their Active Principle.
2. European Patent: EP 0 387 757 A2 Emanuela Maggioni Moratti Use of 5′deoxy-5′methylthioadenosine, S-adenosylmethionine and their salts in the preparation of pharmaceutical compositions favouring hair growth in subjects suffering from baldness, and relative pharmaceutical compositions.
3. U.S. Pat. No. 5,753,213 Emanuela Maggioni Moratti Pharmaceutical Compositions Containing 5′-deoxy-5′-methylthioadenosine S-adenosylmethionine and their Salts for Reducing Seborrhea
4 European Patent: CN101141970 Pablo Villoslada Diaz, et al. Use of 5′-methylthioadenosine (MTA) in the prevention and/or treatment of autoimmune diseases and/or transplant rejection
5. World Patent: WO2007097647 Vern L Schramm, et al. Methods of Treating Cancer
6. European Patent: ES2259552 Pablo Villoslada Diaz, et al. Prevention of auto immune diseases and transplant rejection comprises application of 5′methylthioadenosine and its salts

The mechanism of action is multi-faceted and includes indirect interference by MTA with the demethylation process, one key to cellular breakdown. Additionally, it is toxic to aberrant precancerous and cancerous cells which are commonly deficient in the enzymes necessary to process/metabolize MTA such as the enzyme methylthioadenosine phosphylorase (MTAP).

In mice implanted with human cancer cell linings MTA showed a more than 10-fold efficacy relative to SAMe. This is extremely surprising in light of the above!

A—if MTA is effectively metabolized in the 33.3% range as a metabolite of SAMe
B—if there are alternate therapeutic pathways relative to cancer attributable only to SAMe
Then this efficacy ratio should be well below the 10:1 cited and probably closer to 2 or 3 to 1! In the absence of inhibiting interactions it seems likely that effective metabolism of SAMe to MTA is less than believed and certainly less than may be required.

In either case, MTA would therefore be the dietary supplement of choice for the promotion of cellular integrity and normal growth. The additional advantages are that it would be more-cost effective and better tolerated as MTA does not require the high percentage (45-50%) acid stabilizing salts. This mitigates the GI problems that such necessarily high SAMe doses may cause. These points are novel as well.

Accordingly, MTA and its pharmaceutically acceptable salts, both organic and inorganic, such as sulphoxide, sulfate, butanedisulfonate, p-toluene sulfonate, Hydrochloride etc are dietary supplements, which promote cellular integrity and destroy many aberrant cells at various stages of their development and can prevent their metastasis.

### Example 1

Oral dose of MTA consisting of 600 mg MTA (2 millimoles) in a film-coated tablet as a dietary supplement

### Example 2

Oral dose of MTA consisting of 300 mg of active MTA (mMOL) as sulphoxide, a scavenger of precancerous cells

### Example 3

Oral dose of MTA consisting of 450 mg active MTA (1.5 mMOL) as a p-toluenesulfonate used to mitigate the spread of the cancer by way of demethylation inhibition and MTAs toxicity to cellular deficient cells common found in metastasizing cancer cells.

### OTHER REFERENCES

1. Guildor P, Galli-kienle M, et al. Transmethylation, transsulfuration and amino propylation reactions of S-adenosyl-L-methionine in Vivo. J Biol Chem 1984 Apr. 10; 259(7):4205-11.
2. Avila M A, Garcia-Trevijano E R, et al. Methylthioadenosine. Int J Biochem Cell Biol. 2004 November; 36(11):2125-30.
3. Kamatani N, Kobota M, et al. 5′-Methylthioadenosine is the major source of adenine in human cells. Adv Exp Med Biol. 1984; 165 Pt B: 83-8.
4. Moratti E. Pharmaceutical compositions containing 5′-deoxy-5′methylthioadenosine, S-adenosyl-methionine and their salts for reducing seborrhea. Moratti U.S. Pat. No. 5,753,213. Filed Mar. 13, 1990; Issued May 19, 1998.
5. Methylthioadenosine beneficial in multiple sclerosis. Review of Moreno article methylthioadenosine reverses brain auto immune disease. Nature. October 2006 Vol 2 No 10 pg 522.
6. Stramentinoli G. Pharmacologic Aspects of S-Adenosyl-Methionine. Ann J Med. 1987 Nov. 20; 83(5A):3542.
7. Schlenk F. Methylthioadenosine. Advances in Enzymology and Related Areas of Molecular Biology. Vol 54:195-265.
8. Grillo M A, Colombatto S. S-adenosylmethionine and its products. Amino Acids. 2008 February; 34(2):187-93 Epub 2007 March 3.
9. Moreno B, Hevia H, et al. Methylthioadenosine reverses brain autoimmune disease. Ann Neurol. 2006 September; 60(3):323-34.
10. Judde J G, Ellis M, Frost P. Biochemical analysis of the role of trans methylation in the methionine dependence of tumor cells. Cancer Res. 1989 Sep. 1; 49(17):4859-65.
11. Zappia et al. Enzyme deficiency and tumor suppressor cegenes; absence of MTAP in human tumors. Exp Med Biol 1993; 348:31-423.
12. Guo Y, et al. Regulation of DNA methylation in human breast cancer. J Biol Chem 2002; 277(44):41571-41579.
13. Detich N, et al. The methyl donor S-Adenosylmethionine inhibits active demethylation of DNA. J Biol Chem 2003; 278(23):20812-20820.
14. Shukeir N, et al. Alteration of the methylation status of tumor promoting genes decreases prostate cancer cell invasiveness tumorigenesis in vitro and in vivo. Cancer Res 2006; 66(18)1-9.
15. Stramentinoli G. Intestinal absorption of S-Adenosylmethionine. Pharm Exp Ther 1979; 209(3)323-326.
16. Grillo, et al. S-Adenosylmetionine and its products. Amino Acids 2008; 34(2):187-193.
17. Poirier L, et al. Chemical causation of methyl deficiency and its attendant pathologies. Reg Res Path 2003; 3(1).
18. Bottiglieri, et al. S-Adenosylmetionine in the clinical practice of psychiatry, neurology and internal medicine. Clin Prac Alt Med 2000; 1(4).
19. Kamatani N, et al. Selective killing of human malignant cell lines deficient in methylthioadenosine phosphorylase, a purine metabolic enzyme. Nat Ac Science 1981; 78(2):1219-1223.
20. Moratti E. Use of 5′-deoxy-5′methylthioadenosine, S-adenosyl-methionine and their salts in the preparation of pharmaceutical compositions favouring hair growth in subjects suffering from baldness, and relative pharmaceutical compositions. Moratti patent EP0387757A2. Filed Mar. 12, 1990; Issued Sep. 19, 1990.
21. Stramentinoli G, Gennari F. Adenosine derivatives of anti-inflammatory and analgesic activity, and therapeutic compositions which contain them as their active principle. Stramentinoli U.S. Pat. No. 4,454,122. Filed Aug. 6, 1982; Issued Jun. 12, 1984.
22. Villoslada D, et al. Use of 5′-methylthioadenosine (MTA) in the prevention and/or treatment of autoimmune diseases and/of transplant rejection. Villoslada patent CN101141970. Filed Mar. 12, 2008.
23. Chandan G, et al. Methods of treating cancer. Chandan patent WO2007097647. Filed Aug. 30, 2007.
24. Villoslada D, et al. Prevention of autoimmune diseases and transplant rejection comprises application of 5′-methylthioadenosine and its salts. Villoslada patent ES2259552. Filed Sep. 1, 2006.

1. The use of 5-deoxy-5-methylthioadenosine, and its pharmaceutically acceptable salts as a dietary supplement

2. The use of 5-deoxy-5-methylthioadenosine, and its pharmaceutically acceptable salts as an ingredient contained in a dietary supplement

3. The use as claimed in claims 1 and 2, in compositions that are suitable for oral administration.

4. The use as claimed in claims 1-3, characterized in that the quantity of active principle used is between 1 mg and 5000 mg.

5. The use as claimed in claim 4, characterized in that the quantity of active principle used is preferably between 50 mg and 1200 mg.

6. The use of 5-deoxy-5-methylthioadenosine, and its pharmaceutically acceptable salts in the prevention of cancer.

7. The use of 5-deoxy-5-methylthioadenosine, and its pharmaceutically acceptable salts as an ingredient used in the prevention of cancer.

8. The use as claimed in claims 1 and 2, in compositions that are suitable for oral administration in the prevention of cancer.

9. The use as claimed in claims 1-3, characterized in that the quantity of active principle used is between 1 mg and 5000 mg in the prevention of cancer.

10. The use as claimed in claim 4, characterized in that the quantity of active principle used is preferably between 50 mg and 1200 mg in the prevention of cancer.

11. The use of 5-deoxy-5-methylthioadenosine, and its pharmaceutically acceptable salts in the prevention of Cancer metastasis.

12. The use of 5-deoxy-5-methylthioadenosine, and its pharmaceutically acceptable salts as an ingredient used in the prevention of Cancer metastasis.

13. The use as claimed in claims 1 and 2, in compositions that are suitable for oral administration in the prevention of Cancer metastasis.

14. The use as claimed in claims 1-3, characterized in that the quantity of active principle used is between 1 mg and 5000 mg in the prevention of Cancer metastasis.

15. The use as claimed in claim 4, characterized in that the quantity of active principle used is preferably between 50 mg and 1200 mg in the prevention of Cancer metastasis.
